(19) United States
(12) Patent Application Publication
(10) Pub. No.: US 2015/0141948 A1
(43) Pub. Date: May 21, 2015

(54) ABSORBENT ARTICLE WITH COLOR MATCHED SURFACES

(71) Applicant: The Procter & Gamble Company, Cincinnati, OH (US)

(72) Inventors: Charles John BERG, Jr., Wyoming, OH (US); Margaret Henderson HASSE, Wyoming, OH (US)

(21) Appl. No.: 14/541,747

(22) Filed: Nov. 14, 2014

Related U.S. Application Data
(63) Continuation of application No. 12/848,344, filed on Aug. 2, 2010.
Application No. 12/848,344 is a continuation of application No. 10/902,819, filed on Jul. 30, 2004, now abandoned.

(51) Int. Cl. A61F 13/56; A61F 13/514; A61F 13/511

(52) U.S. Cl. CPC A61F 13/5616; A61F 13/511; A61F 13/514; A61F 13/51496
USPC 604/385.04

(57) ABSTRACT

A disposable absorbent article having a backsheet, a topsheet and an absorbent element. The topsheet, backsheet, and the absorbent element has an imparted color in which each of the imparted colors for the aforementioned elements are color matched. Color matching exists when the colors are contained within a specified CIELab color space volume, have a specified hue difference, or have a total color difference.

### FIELD OF THE INVENTION

This invention is directed to disposable absorbent articles, such as diapers, training pants, adult incontinence articles, feminine protection articles and the like wherein the article comprises elements that are quantitatively color matched according to International Commission on Illumination L*a*b* system (hereinafter “CIELab” from the French title Commission Internationale de l'Eclairage) to achieve a harmonic flow of colors in the article.

### BACKGROUND OF THE INVENTION

Absorbent articles such as disposable diapers, pull-on diapers, training pants, sanitary napkins, pantiliners, incontinence briefs, and the like, are well known in the art and are highly effective for absorbing and containing urine and other bodily exudates. Since their introduction into the marketplace, absorbent articles have continued to improve regarding fit and functionality. For example, sanitary napkins are constructed to provide a better fit with the wearer of the article and contain larger quantities of bodily exudates without adverse impact to the wearer's skin.

However, feminine protection articles have not advanced quite as significantly in regard to aesthetic design. Since the inception of commercially available feminine protection articles, the feminine protection articles' predominant color is that which is inherent within the materials selected for construction. The materials are generally a white color often achieved by the use of pigments such as titanium dioxide or by bleaching. The white color has historically been common to absorbent articles, in general including diapers, adult incontinence products, underarm sweat products, collar inserts, sanitary napkins and pantiliners to communicate a hygienic condition. This did fit well with the historically predominant undergarment color in which these articles are worn, namely white undergarments. As a result white pantiliners or white sanitary napkins are not easily recognizable when used in such white undergarments.

Very recently fashion has led women more frequently to use other colors than white undergarments. This has been dictated not only by fashion itself but also as a result of the development of clothing which has a certain translucency and allows the color of the undergarment to be recognized. Furthermore very recently so-called G-String undergarments or “Tanga-Slips” have become widely used in particular by a younger generation women. These so-called string tangas are particularly used to prevent easy recognition of the contour of the undergarment through tight fitting clothes.

It has thus become a need to have sanitary napkins or pantiliners available which match the color of the undergarments (and typically the garments) in order to prevent a color-based recognition of the presence of such articles (under the assumption that modern clothing can be translucent).

For sanitary napkins or pantiliners having so-called wings which are folded around the outside of the undergarment this is even more important since the wing part of the sanitary napkin or pantiliner is on purpose folded onto the outside of the undergarment and therefore easily visible, depending on the clothing worn over the undergarment.

To provide the appealing aesthetic design of a colored sanitary napkin or pantiliner, elements of the feminine protection article must be matched. Ensuring that colors within a feminine protection article match is important but difficult to achieve. A feminine protection article having matching colors communicates to the consumer that the product is of high quality and differentiates the product from competitors' products. A feminine protection article having matching colors is holistically pleasing and is believed to be preferred by consumers. However, absorbent articles are difficult products to color match. Feminine protection articles are comprised of a variety of materials. Printing, coating, or impregnating are well known methods for imparting color, but not all materials used in feminine protection article construction are amenable to each of these methods. As a result, one material may only be colored by printing or coating while a different material adjacent to the first may only accept impregnation of color. Even if two different materials are amenable to a single method for imparting color, the imparted color may appear mismatched between the two materials in the finished product. Many surfaces of a feminine protection article are laminates whereby the color-bearing layer is covered by a second layer. The resulting laminate exhibits a muted color compared to the color of the color-bearing layer. Furthermore, the texture of the material may also adversely impact the color imparted.

A further problem in color matching is determining when two surfaces have matching colors. Reliance on human discernment of color differences or matching is undependable because color differentiation is a highly variable personal characteristic. As a result, what appears to be color matched to one observer may be distinguishable to another. Furthermore, quantifying the highly qualitative concept of human discernment of color differences or matching between two or more surfaces is very difficult.

### SUMMARY OF THE INVENTION

In response to the difficulties and problems described herebefore, a disposable absorbent article is disclosed having color matched elements according to CIELab color space calculations. Specifically, a disposable absorbent article is disclosed comprising a backsheet, topsheet, and an absorbent element comprising an externally visible surface comprising an imparted color. The imparted colors may be contained within a CIELab color space volume of less than about 190, more preferably less than about 150, and most preferably less than about 100. The imparted colors may be contained within a CIELab total color difference of less than about 3, preferably less than 2. The imparted colors may be contained within a CIELab color space hue difference of less than about 4, preferably less than 2, and most preferably less than 1.

Additionally, an element may further comprise a laminate material wherein the laminate material comprises at least one lamina exhibiting an imparted color and wherein the imparted color of the lamina and the imparted color of the element are not color matched.

### BRIEF DESCRIPTION OF THE DRAWINGS

FIG. 1 is an illustration of the Commission Internationale de l'Eclairage L*a*b* color space.

FIG. 2 is a top plan view of a sanitary napkin embodiment of the present invention with portions cut away to reveal the underlying structure of the sanitary napkin.

### DETAILED DESCRIPTION OF THE INVENTION

The present invention matches the imparted colors of at least two elements of the sanitary napkin within a color space volume of less than about 190. Namely, the two elements can be the topsheet, the backsheet, and the absorbent element. Optionally, the release liner and the adhesive located on the backsheet can be elements.

“Absorbent articles” as referred to herein are primarily sanitary napkins, pantiliners, or incontinence pads or any other type of article that can be worn in the crotch region of an undergarment. However, articles such as sweat-absorbent underarm pads, nursing pads or collar inserts can also benefit from the present invention. Baby diapers, adult incontinence diapers, and human waste management devices may benefit from the present invention even though they are conventionally not worn in conjunction with an undergarment.

As used herein, the term “element” refers to the separate and discrete parts that must be united together to form the absorbent article. Each element may comprise one or more lamina. Furthermore, elements may share a common, continuous lamina that has not been subjected to a coloration technique yielding an imparted color. However, elements may not share a common, continuous lamina that has been subjected to a coloration technique yielding an imparted color.

The term “disposable” is used herein to describe absorbent articles that are not intended to be laundered or otherwise restored or reused as absorbent articles (i.e., they are intended to be discarded after a single use and, preferably to be recycled, composted or otherwise disposed of in an environmentally compatible manner).

As used herein, the term “garment-facing surface” refers to a surface oriented opposite the wearer-facing surface. The garment-facing surface is the portion of the absorbent article which attaches to the user's undergarment. The garment-facing surface can comprise a fastening adhesive for attachment. Generally, in the case of sanitary napkins, pantliners or thongliners a so-called panty fastening adhesive is preferred to be present on the backsheet for attachment to an undergarment. However, for sweat pads, e.g. underarm sweat pads, either attachment to an adjacent garment or attachment to the skin of the wearer directly can also be considered.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

As used herein, the term “externally visible surface,” refers to a visible surface on either side of the absorbent article that a human viewer may visually discern with the unaided eye (excepting standard corrective lenses adapted to compensate for near-sightedness, farsightedness, or astigmatism) in standard lighting conditions from a point of reference of viewing the article before proper application. The externally visible surface (i) is visually discernible without tearing, ripping, puncturing, or otherwise mutilating or damaging the article and (ii) has been subjected to a coloration technique resulting in an imparted color. Preferably, the externally visible surface of an element may be color matched according to the present invention.

As used herein, the term “wearer-facing surface,” refers to the portion of the absorbent article which faces the user of the article during use and is liquid pervious in order to allow liquids to pass into the absorbent article.

As used herein, the term “imparted colors” are those colors printed, coated, or impregnated onto or into the externally visible surface of the sanitary napkin. Imparted colors are those colors with a CIELab C* (formula provided below) of greater than about 6.

As used herein, “standard lighting conditions” refer to lighting conditions in which human vision operates efficiently (e.g., the human eye is able to discern complex patterns, shading, and colors). Specifically, for the purposes of describing this invention, standard lighting conditions are at least one of the following:
a) natural illumination as experienced outdoors during daylight hours,
b) the illumination of a standard new (i.e. not used) 100 watt incandescent white light bulb at a distance of 2 meters from the object to be illuminated with no intervening articles (e.g. lamp shade), or
c) as defined by CIE D65 standard illuminant lighting at 800 lux to a 1964 10 degree CIE standard observer.

As used herein, the term “color matched” refers to colors that fall within a prescribed color space volume, that have a prescribed color space hue difference, and/or that have a prescribed total color difference.

The term “color,” as used herein, is a visual effect resulting from the eye's ability to distinguish the different wavelengths or frequencies of light. The apparent color of an object depends on the wavelength of the light that it reflects. While a wide palette of colors can be employed herein, it is preferred to use a member selected from the group consisting of orange, purple, lavender, red, green, blue, yellow, and violet.

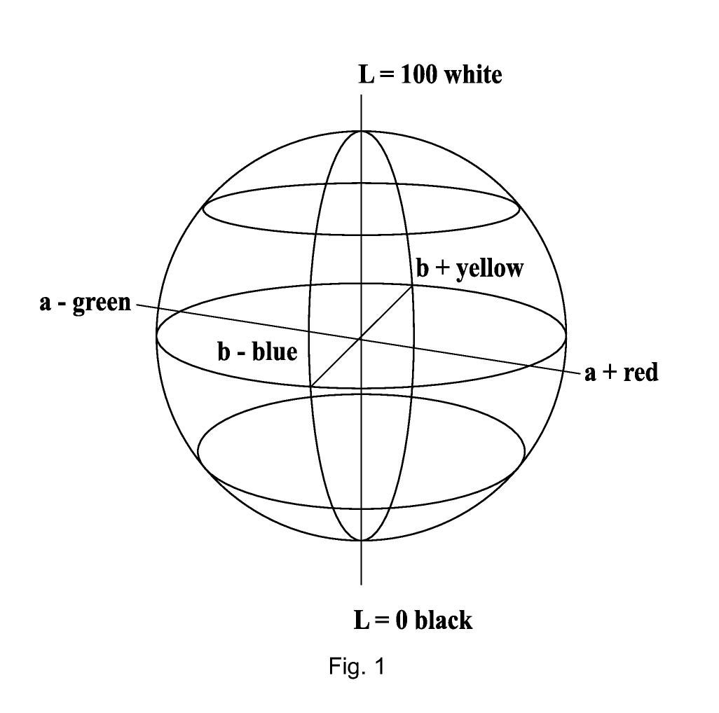

Referring to FIG. 1, a sanitary napkin is generally constructed from a variety of elements that are joined together. The various elements of sanitary napkin construction generally have at least one externally visible surface. It is desirable for the sanitary napkin to comprise a topsheet, a backsheet, and an absorbent element. A surface initially not an externally visible surface may subsequently become an externally visible surface during storage, application, wear, or disposal. For example, a release liner may be removed from the adhesive placed on the garment facing surface. The removal of the release liner may reveal an externally visible surface on the adhesive. Generally, the elements of the sanitary napkin will contain two surfaces typically oriented with a wearer-facing surface and a garment-facing surface. Color matching may occur between the wearer-facing surface, garment-facing surface, or both. In one embodiment, color matching will occur between the externally visible surfaces of the elements including the release liner.

The various colors exhibited by the sanitary napkin may generally be divided into two categories. Inherent colors are typically colors with little to no chroma. Qualitatively, inherent colors represent the white to off-white colors traditionally associated with sanitary napkins. The inherent color is usually the color of the raw material that comprises the externally visible surfaces. The whiteness of the material may be manipulated, such as by bleaching, printing, coating, or impregnating a substrate with titanium dioxide pigment. More specifically and quantitatively, inherent colors are those colors with a CIELab chroma value, C*, of less than about 6 (formula provided below). Imparted colors are those colors printed, coated, or impregnated onto or into the externally visible surface of the sanitary napkin. Imparted colors are those colors with a CIELab C* of greater than about 6. Externally visible surfaces generally contain an inherent color and may contain one or more imparted colors.

It is further recognized that an externally visible surface having an imparted color may comprise a single stratum or may be a laminate comprising more than one lamina. Regarding a single stratum, the imparted color is imparted onto or within that stratum. Regarding a laminate, color may be imparted on one or more of the lamina. For example, a laminate generally may comprise an exterior lamina (i.e., the layer closest in proximity within the line of sight of an observer) and at least one interior lamina. One or more of the lamina may have an imparted color. For example, the laminate may comprise an exterior lamina with no imparted color and at least one interior lamina with an imparted color. When the sanitary napkin is viewed, the imparted color of the interior lamina provides the coloration for the laminate as a whole. Thus, the perceptible color of the laminate as a whole is the result of the exterior lamina and other optional interior laminae possibly diffusing and shifting the imparted color of the interior lamina. As a result, the imparted color of the laminate as a whole may be different (i.e., not color matched) as compared to the imparted color of the individual lamina if viewed in isolation. Additionally, the exterior lamina and one or more interior laminae may exhibit imparted colors such that the imparted color of the individual lamina when viewed in isolation are not color matched with the imparted color of the laminate as a whole.

The imparted color on an externally visible surface may be provided by a variety of coloration techniques that are well known in the art. Color may be provided to an externally visible surface by coloration techniques including, but not limited to, printing, coating, and impregnating. Various printing methods may be used to impart color including, but not limited to, letterpress, flexography, gravure, offset lithography, screen, and inkjet. All methods are well known in the art.

Letterpress, the oldest method of printing, involves ink or other equivalent material being applied to the top of a raised surface. This surface is pressed against a substrate, thus transferring the ink to the substrate. Flexographic printing uses a printing plate, often cylindrical, made of rubber, plastic, or other flexible material. Ink is applied to a raised image on the plate. The plate is then placed in contact with a substrate, and ink is transferred to the substrate. Water-based and solvent-based inks are used in flexography. Most inks used are fast drying which makes flexography particularly well-suited for printing on plastics, foils, compressible surfaces, and other nonabsorbent substrate.

Gravure printing uses a print cylinder having depressions of varying depths that are etched into the cylinder. This method of printing is performed by partially immersing the etched cylinder (generally about a fourth of the cylinder diameter) into an enclosed fountain or trough of ink. The etched cells, which produce the image, are filled with ink, and the surface of the cylinder also becomes coated with ink. Since the surface of the cylinder is non-image producing, ink is not desirable on the cylinder surface. This undesired ink is removed by a doctor blade or knife which wipes all of the surface ink from the cylinder. As the printing cylinder comes in contact with the substrate, the ink contained within the cells is transferred to the substrate. Gravure is ideal for continuous printing operations and the printing of very long runs. Generally, solvent-based inks are used in gravure printing.

Lithographic printing, or offset lithography, is a printing method that utilizes surface characteristics on an image carrying offset plate. Offset plates are typically made from a thin paper, plastic, or a metal sheet which once exposed and processed can be wrapped around a cylinder of a press for printing. The offset plate contains two areas: an image area that is hydrophobic and a non-image area that is hydrophilic. While the basic principle is common, there are many differences between offset plates and the method they use to separate the image from the non-image areas. Generally, ink adheres to the hydrophobic image area while being repelled from the hydrophilic non-image area. The ink and watered offset plate may be printed on a second cylinder usually coated in rubber. The second cylinder then off-sets this ink and water impression onto the substrate.

Screen printing utilizes a porous screen made from silk or other polymeric material. The screen is attached to a frame. A stencil is produced on the screen either photo-mechanically or manually. The non-printing areas are protected by the stencil. Printing is done on the substrate under the screen by applying a viscous ink to the screen. The ink is forced through the fine openings of the screen with a rubber squeegee or roller.

Inkjet printing is a non-impact dot-matrix technology where ink droplets are jetted from a small aperture directly to specified positions on a medium to create an image Inkjet printing may be done on a continuous method or a drop-on-demand method. Continuous inkjet printing involves a continuous stream of ink droplets. Generally, the ink droplets may be charged by a charge electrode. If the droplets are not charged, the droplet travels directly to the substrate through and unimpeded by a voltage carrying plate. Droplets that are charged are deflected by the voltage carrying plate. If diverted, the droplet is captured and recirculated prior to reaching the substrate. Another continuous inkjet method charges all droplets and the voltage plate controls droplet placement onto the substrate or diversion. Drop-on-demand inkjet printing, as the name implies, provides an ink droplet only when needed. Droplets are formed by a variety of methods with thermal and piezoelectric drop formation being most common. Thermal inkjet printing involves the ink droplets being expelled from a nozzle by the rapid expansion of an ink vapor bubble created by a small heater. Piezoelectric inkjet printing involves the ink droplets being expelled from a nozzle by a pressure wave created from the expansion of a piezoelectric ceramic upon application of a voltage Inkjet printing techniques are well known in the art as described in Hue. P. Le, Progress and Trends in Ink-Jet Printing Technology, Journal of Imagining Science and Technology, Vol. 42, pages 49-62.

A variety of coating techniques may be used to impart color onto an externally visible surface. Suitable coating techniques are well-known in the art and include, but are not limited to, bead extruders, slot die coaters, spray nozzles, dip tanks, brushes, and combinations thereof. Suitable slot die coaters include the EP11 Applicator available from Nordson Corp., Dawsonville, Ga. or the MR1300 Slot Die Coater available from ITW Dynatec Americas, Hendersonville, Tenn. Suitable coatings include, but are not limited to, adhesives, varnishes, latexes, lotions, waxes, and paraffins. The coatings generally will contain a dye, pigment, or combination.

Color may be imparted to an externally visible surface by way of impregnation of a colorant into a substrate or by limited coating onto a substrate surface. Colorants such as dyes, pigments, or combinations may be impregnated in the formation of substrates such as polymerics, resins, or nonwovens. For example, the colorant may be added to molten batch of polymer during film, fiber, or filament formation.

Referring to FIG. 1, the imparted color is matched according to the Commission Internationale de l'Eclairage L*a*b* color space (hereinafter “CIELab”). CIELab is a mathematical tristimulus color scale based on the CIE 1976 standard. CIELab allows colors to be described quantitatively and with precision. As presented in FIG. 1, CIELab allows a color to be plotted in three-dimensional space analogous to the Cartesian xyz space. CIELab has the colors green to red on what is traditionally the x-axis in Cartesian xyz space. CIELab identifies this axis as the a-axis. A negative a* value represents green and a positive a* value represents red. CIELab has the colors blue to yellow on what is traditionally the y-axis in Cartesian xyz space. CIELab identifies this axis as the b-axis. Negative b* values represent blue and positive b* values represent yellow. CIELab has lightness on what is traditionally the z-axis in Cartesian xyz space. CIELab identifies this axis as the L-axis. The L*-axis ranges in value from 100, which is white, to 0, which is black. An L* value of 50 represents a mid-tone gray (provided that a* and b* are 0). Any color may be plotted in CIELab according to the three values (L*, a*, b*).

The three-dimensional CIELab allows the three color components of chroma, hue, and lightness to be calculated. Within the two-dimensional space formed from the a-axis and b-axis, the components of hue and chroma can be determined. Chroma is the relative saturation of the perceived color and is determined by the distance from the origin as measured in the a*b* plane. Chroma, for a particular (a*, b*) set is calculated according to the following formula: C*=\sqrt{a*²+b*²}. For example, a color with a*b* values of (10,0) would exhibit a lesser chroma than a color with a*b* values of (20,0). The latter color would qualitatively be perceived as being more red than the former. Hue is the relative red, yellow, green, and blue in a particular color. A ray can be created from the origin to any color within the two-dimensional a*b* space. Hue is the angle measured from 0° (the positive a*-axis) to the created ray. Hue can be any value of between 0° to 360°. Lightness is determined from the L* value with higher values being more white and lower values being more black.

The testing and comparison of externally visible surfaces occurs between discrete elements of sanitary napkin construction each having at least one externally visible surface. A permissible comparison would be, for example, between the externally visible surface of the topsheet and the externally visible surface of the absorbent element. A comparison should not be done between like elements. For example, a comparison should not be done between two sample regions on a single element of sanitary napkin construction (e.g., a comparison between two points on a backsheet). Externally visible surfaces were tested for reflective color utilizing the following standardized procedure.

Color matching of two or more elements comprising an externally visible surface, wherein each externally visible surface comprises an imparted color, can be determined by color space volume, total color difference, and/or hue difference.

i. Color Space Volume

One embodiment of the present invention is color matching of two or more externally visible surfaces of differing elements such that the imparted colors occupy a defined CIELab color space volume. In other words, the color space volume represents how matchable one or more colors are to one another. The match is defined by the boundary surface and depends on the position of the color in the color space. Characterizing color matching within a volume is desirable such that the volume accounts for and considers all three dimensions within CIELab. While not being limited to the theory, such a three-dimensional measurement is believed to more fully characterize the difference in two colors. The CIELab color space volume (V), for a first color (L*₁, a*₁, b*₁) and a second color (L*₂, a*₂, b*₂), is calculated according to the following formula:

$$V = \frac{4}{3} π \vert \frac{Δ L *}{2} \vert \vert \frac{Δ a *}{2} \vert \vert \frac{Δ b *}{2} \vert .$$

Within the formula, ΔL* is the difference in L* values between the two colors and is calculated by: ΔL*=L*₂−L*₁. The Δa* is the difference in a* values between the two colors and is calculated by: Δa*=a*₂−a*₁. The Δb* is the difference in b* values between the two colors and is calculated by: Δb*=b*₂−b*₁. The CIELab color space volume results in a solid substantially ellipsoidal in shape; however, if ΔL*, Δa*, and Δb* are equal, the solid will be spherical. As used herein, a “solid” refers to the mathematical concept of a three-dimensional figure having length, breadth, and height (or depth). An ellipsoidal volume is preferred to calculate volume because an ellipsoid generally requires the dimensional differences of ΔL*, Δa*, and Δb* to be relatively more uniform than other solids. Furthermore, it is believed that ellipsoidal volumes are more visually acceptable (i.e., less detectable color mismatch by human perception) than spherical volumes.

Ideally, the imparted colors of at least two externally visible surfaces of discrete elements will occupy a CIELab color space volume of less than about 190. The externally visible surfaces are analyzed according to the Test Method described below. Upon analysis, the inherent color of an element comprising an externally visible surface will yield L*, a*, and b* coordinates. The CIELab color space volume is then calculated utilizing the formula presented above. The resulting volume will preferably be less than about 190. More preferably, the resulting volume will be less than about 150. Even more preferably, the resulting volume will be less than about 100.

It should be recognized that the imparted colors of more than two discrete elements having an externally visible surface may occupy the aforementioned CIELab color space volumes. In calculating the color space volume for more than two elements, volume is calculated using the maximum and minimum L*, a*, and b* from a set of elements. A given set of elements will yield a set of L*, a*, and b* values. A maximum color value is selected by taking the maximum L*, the maximum a*, and the maximum b* from the set of L*, a*, and b* values. Likewise, a minimum color value is selected by taking the minimum L*, the minimum a*, and the minimum b* from the set of L*, a*, and b* values. The maximum color values and minimum color values are used to calculate V according to the formula presented above. Preferably, the imparted colors of more than two discrete elements having an externally visible surface will occupy the volume. More preferably, the imparted colors of more than three discrete elements having an externally visible surface will occupy the volume.

ii. Color Space Total Difference

One embodiment of the present invention is color matching of externally visible surfaces such that the colors have a specified CIELab color space total color difference (ΔE). In other words, this is a single number that expresses the magnitude of the difference between two colors. The value tells nothing about the nature of the color difference. Unlike the measurement of color space volume which can measure more than two colors, the total color difference measurement only can measure two colors. Characterizing color matching by the total color difference is desirable in that total color difference accounts for and considers all three dimensions within CIELab. While not being limited to this theory, such a three-dimensional measurement is believed to more fully characterize the difference in two colors. The total color difference represents the distance between two points within CIELab color space. The CIELab color space total color difference (ΔE) for a first color (L*₁, a*₁, b*₁) and a second color (L*₂, a*₂, b*₂), is calculated according to the following formula: ΔE=\sqrt{(ΔL*)²+(Δa*)²+(Δb*)²}. Within this formula, ΔL* is the difference in L* values between the two colors and is calculated by: ΔL*=L*₂−L*₁. The Δa* is the difference in a* values between the two colors and is calculated by: Δa*=a*₂−a*₁. The Δb* is the difference in b* values between the two colors and is calculated by: Δb*=b*₂−b*₁.

Ideally, at least two elements comprising an externally visible surface each comprising an imparted color will have a CIELab color space total color difference of less than about 3. The externally visible surfaces are analyzed according to the Test Method described below. Upon analysis, the inherent color of an element comprising an externally visible surface will yield L*, a*, and b* coordinates. The CIELab color space total color difference is then calculated utilizing the formula presented above. The resulting ΔE will preferably be less than about 3. Even more preferably, the resulting ΔE will be less than about 2.

It should be recognized that imparted colors of more than two discrete elements having an externally visible surface may occupy the aforementioned CIELab color space total color difference. Preferably, the imparted colors of more than two discrete elements having an externally visible surface will be less than the aforementioned ΔE. More preferably, the imparted colors of more than three discrete elements having an externally visible surface will be less than the aforementioned ΔE. Comparing more than two elements implies that, no matter which two elements having an externally visible surface containing the imparted color are compared, the resulting total color difference will be less than the aforementioned ΔE values.

In one non-limiting example, the color for the topsheet 21 is green and the color for the backsheet 22 is green. The parameters for ΔL*, Δa*, and the Δb* are (0.35, 0.30, 0.20). The ΔE that results from this example is 0.50.

iii. Color Space Hue Difference

One embodiment of the present invention is color matching of externally visible surfaces such that the colors have a specified CIELab color space hue difference (AH). Characterizing color matching by the hue difference is desirable in that hue difference accounts for and considers all three dimensions within CIELab. While not being limited to this theory, such a three-dimensional measurement is believed to more fully characterize the difference in two colors. The hue difference represents the distance between two points within CIELab color space. The CIELab color space hue difference (AH) for a first color (L*₁, a*₁, b*₁) and a second color (L*₂, a*₂, b*₂), is calculated according to the following formula: ΔH=\sqrt{(ΔE)²−(ΔC)²−(ΔL*)²}. Within this formula, ΔE is the CIELab color space total color difference between the two colors and is calculated as presented above. The ΔC is the CIELab color space chroma difference between the two colors and is calculated by: ΔC=\sqrt{a*₂²+b*₂²}−\sqrt{a*₁²+b*₁²}. The ΔL* is the difference in L* values between the two colors and is calculated by: ΔL*=L*₂−L*₁

Ideally, at least two externally visible surfaces each comprising an imparted color will have a CIELab color space hue difference of less than about 4. The externally visible surfaces are analyzed according to the Test Method described below. Upon analysis, the inherent color of an element comprising an externally visible surface will yield L*, a*, and b* coordinates. Two elements are selected and the L*, a*, and b* values of the elements are inserted into the formula presented above to result in a hue difference. The resulting hue difference will preferably be less than about 4. More preferably, the resulting ΔH will be less than about 3. Most preferably, the resulting ΔH will be less than about 1.

It should be recognized that imparted colors of more than two discrete elements having an externally visible surface may occupy the aforementioned CIELab color space hue difference. Preferably, the imparted colors of more than two discrete elements having an externally visible surface will be less than the aforementioned ΔH. More preferably, the imparted colors of more than three discrete elements having an externally visible surface will be less than the aforementioned ΔH. Comparing more than two elements implies that, no matter which two elements having an externally visible surface containing the imparted color are compared, the resulting hue difference will be less than the aforementioned ΔH values.

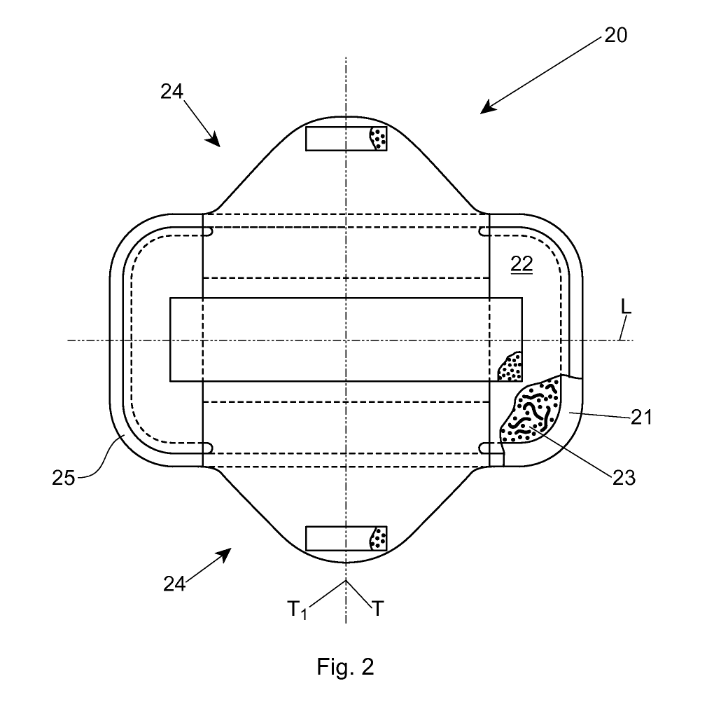

FIG. 2 shows a plan view of a sanitary napkin 20 of the present invention. The absorbent article according to the present invention is conventionally constructed of three main elements: the topsheet 21, the backsheet 22, and the absorbent element 23 disposed between the topsheet 21 and backsheet 22. The topsheet 21 faces the user of the article during use and is liquid pervious in order to allow liquids to pass into the sanitary napkin 20. The topsheet 21 can be an apertured topsheet 21. The backsheet 22 provides liquid containment such that absorbed liquid does not leak through the article. The backsheet can be joined about the periphery 25. Optionally, sanitary napkin 20 can have side extensions, commonly referred to as “wings” 24 designed to wrap the sides of the crotch region of the panties of the user of sanitary napkin 20. Sanitary napkins 20, including topsheets 21 for use as the body facing surface thereof, are well known in the art and need no detailed description of various alternative and optional designs.

Many absorbent articles and constructions, including particular materials, are known in the art and have been described in ample detail over time. All of such materials are useful in the context of the present invention, provided that they meet the color requirement as defined herein by the CIELab values. According to the present invention the topsheet 21, the backsheet 22, and the absorbent element 23 have a CIELab value of volume of less than 190. Typically this will require only moderate modification of the material composition while maintaining the majority of the conventional material characteristics. In the following, examples of materials, which are particularly beneficial for the use in the absorbent articles according to the present invention, are mentioned. Those skilled in the art will readily be able to identify alternative materials, which can also be used in the context of the absorbent articles according to the present invention.

The CIELab value, utilized herein to define the darkness/lightness of the materials of the absorbent articles according to the present invention, are units of color measurement in the aforementioned CIELab system. The absorbent articles herein, and hence the materials of which the absorbent articles are made of, might be of any color provided that the CIELab value defined herein is met.

Ideally, the imparted colors for at least two of the elements of the sanitary napkin 20 will occupy a CIELab color space volume of less than about 190. Again, the elements of the sanitary napkin 20 are the topsheet 21, the backsheet 22, and the absorbent element 23. The externally visible surfaces are analyzed according to the Test Method described above. Upon analysis, the inherent color of an element comprising an externally visible surface will yield L*, a*, and b* coordinates. The CIELab color space volume is then calculated utilizing the formula presented above. The resulting volume will preferably be less than about 190. More preferably, the resulting volume will be less than about 150. Even more preferably, the resulting volume will be less than about 100. Each major element of the sanitary napkin 20 is described below.

### Topsheet

The term apertured topsheet 21 as used herein refers to topsheets 21 having apertures. In general the apertured topsheet is compliant, flexible, soft feeling and non-irritating to the wearer's skin Typically the topsheet 21 comprises a plurality of apertures having a pore size of from about 0.0001 mm to about 5 mm. All apertures might have the same dimensions or apertures of different dimensions might be present. The open area of the apertured topsheet is typically from about 1% to about 50%, preferably from about 5% to about 45%, more preferably from about 10% to about 40% and most preferably from about 20% to about 35%.

The apertured topsheet is typically an apertured polymeric film. Suitable apertured polymeric film topsheets for use herein include polymeric apertured formed films, apertured formed thermoplastic films, apertured plastic films, and hydroformed thermoplastic films; porous foams; reticulated foams; reticulated thermoplastic films; and thermoplastic scrims. Apertured formed films are particularly suitable for use herein as the apertured topsheet because they are pervious to body exudates and yet nonabsorbent and have a reduced tendency to allow fluids to pass back through and rewet the wearer's skin Thus, the surface of the formed film that is in contact with the body remains dry, thereby reducing body soiling and creating a more comfortable feel for the wearer. Suitable formed films are described in U.S. Pat. No. 3,929,135 (Thompson), issued Dec. 30, 1975; U.S. Pat. No. 4,324,246 (Mullane et al.), issued Apr. 13, 1982; U.S. Pat. No. 4,342,314 (Radel et al.), issued Aug. 3, 1982; U.S. Pat. No. 4,463,045 (Ahr et al.), issued Jul. 31, 1984; and U.S. Pat. No. 5,006,394 (Baird), issued Apr. 9, 1991. Particularly preferred microapertured formed film topsheets are disclosed in U.S. Pat. No. 4,609,518 (Curro et al.), issued Sep. 2, 1986 and U.S. Pat. No. 4,629,643 (Curro et al.), issued Dec. 16, 1986.

The body surface of the polymeric film topsheet can be hydrophilic so as to help liquid to transfer through the topsheet faster than if the body surface was not hydrophilic. In a preferred embodiment, surfactant is incorporated into the polymeric materials of the formed film topsheet such as is described in PCT Publication No. WO93/09741, “Absorbent Article Having A Nonwoven and Apertured Film Coversheet” filed on Nov. 19, 1991 by Aziz et al. Alternatively, the body surface of the topsheet can be made hydrophilic by treating it with a surfactant such as is described in the above referenced U.S. Pat. No. 4,950,254.

Other suitable apertured topsheet for use herein are made of woven or nonwoven materials or knit materials. Such materials might be comprised of natural fibers (e.g., wood or cotton fibers), synthetic fibers (e.g., polymeric fibers such as polyester, polypropylene, or polyethylene fibers) or from a combination of natural and synthetic fibers. The apertured topsheet herein can be made from a nonwoven or woven material or a polymeric film. Such films and nonwovens or wovens can be made for example from polymers such as polyethylene or polypropylene compositions. Conventionally such polymers have been provided with a coloring material such as titanium dioxide to provide a white opacity. Simply using other coloring materials (e.g., dyes, pigments and so on) namely coloring materials (like black, navy blue, gray and the like) provides a colored film or nonwoven. The use of other coloring materials instead of titanium dioxide so as to meet the CIELab values for the materials to be used herein, has the advantage to not cause any substantial material change. Also selecting particular coloring materials to be used in the material from which the topsheet can be made also provides the additional benefit of better material characteristics (besides less cost and a reduced environment burden).

It is to be understood herein that any conventional method known to those skilled in the art to provide a colored apertured topsheet 21 (i.e., material meeting the CIELab value requirement herein) is suitable for use herein. Colored topsheets 21 can typically be made of any of the materials mentioned above by different methods well known to those skilled in the art, including pigmenting the materials, dying the materials, or color printing the materials.

### Backsheet

In general, the backsheet 22 is compliant, flexible, and soft feeling. The backsheet 22 prevents the exudates absorbed and contained in the absorbent element 23 from wetting clothes that contact the absorbent article such as undergarments. Preferably the backsheet 22 is impervious to liquids (e.g., menses, sweat and/or urine). It can be manufactured from a thin plastic film, although other flexible liquid impervious materials can also be used. As used herein, the term “flexible” refers to materials that are compliant and will readily conform to the general shape and contours of the human body. The backsheet 22 preferably also can have elastic characteristics allowing it to stretch in one or more directions.

The backsheet can comprise a woven or nonwoven material, polymeric films such as thermoplastic films of polyethylene or polypropylene, or composite materials such as a film-coated nonwoven material or fiber coated film. Conventionally absorbent articles comprise a backsheet of a polyethylene film having a thickness of from about 0.012 mm to about 0.100 mm, more typically from about 0.030 to about 0.060 mm.

The backsheet is preferably breathable, i.e., allows the transmission of water vapor, or even more preferable the transmission of air, however without sacrificing its main purpose to provide leakage protection for absorbed liquids. The backsheet can also comprise more than one breathable layer so as to replace a single breathable backsheet layer by at least 2 or 3 layers of a different or the same material. In particular two breathable layers forming together the breathable backsheet are preferred.

According to the present invention a polyethylene backsheet, or in fact any backsheet made from polymeric material can be provided in any color including dark colored by using a color filler matching the color of interest. For instance, a dark colored backsheet might be provided by using the suitable material among of a color filler/pigments instead of the conventional white materials like titanium dioxide. As will be recognized by those skilled in the art the dark color of the backsheet can be provided in the same manner as in the dark colored topsheet.

### Absorbent Element

Conventionally the absorbent element can be a single entity or comprise several layers. It can include the following components: (a) optionally a primary fluid distribution layer; (b) optionally a secondary fluid distribution layer; (c) a fluid storage layer; (d) optionally a fibrous layer underlying the storage layer; and (e) other optional components.

a. Optional Primary Fluid Distribution Layer

One optional component of the absorbent element according to the present invention is the primary fluid distribution layer. This primary distribution layer typically underlies the topsheet (if present) and is in fluid communication therewith. The primary distribution layer acquires body fluid for ultimate distribution to the storage layer. This transfer of fluid through the primary distribution layer occurs mainly in the thickness, but may also provide distribution along the longitudinal L and transverse T directions of the article.

b. Optional Secondary Fluid Distribution Layer

Also optional according to the present invention is a secondary fluid distribution layer. This secondary distribution layer typically underlies the primary distribution layer and is in fluid communication therewith. The purpose of this secondary distribution layer is to readily acquire bodily fluid from the primary distribution layer and distribute it along the longitudinal and transverse directions of the article before transfer to the underlying storage layer. This helps the fluid capacity of the underlying storage layer to be fully utilized.

c. Fluid Storage Layer

Positioned in fluid communication with, and typically underlying the primary or secondary distribution layers, is a fluid storage layer. The fluid storage layer may be any absorbent means that is capable of absorbing or retaining liquids (e.g., menses and/or urine). The fluid storage layer may be manufactured in a wide variety of sizes and shapes (e.g., rectangular, oval, hourglass, asymmetric, etc.) and from a wide variety of liquid-absorbent materials commonly used in sanitary napkins and other absorbent articles such as comminuted wood pulp that is generally referred to as airfelt. Examples of other suitable absorbent materials include creped cellulose wadding, modified cross-linked cellulose fibres (such as those described in U.S. Pat. No. 5,217,445 issued to Young et al. on Jun. 8, 1993), capillary channel fibres (that is, fibres having intra-fibre capillary channels such as those described in U.S. Pat. No. 5,200,248 issued to Thompson et al. on Apr. 6, 1993), absorbent foams (such as those described in U.S. Pat. No. 5,260,345, issued to DesMarais et al. on Nov. 9, 1993 and U.S. Pat. No. 5,268,244 issued to DesMarais et al. on Dec. 7, 1993), thermally bonded airlaid materials (such as those material described in U.S. Pat. No. 5,607,414, issued to Richards et al. on Mar. 4, 1997), absorbent sponges, synthetic staple fibres, polymeric fibres, hydrogel-forming polymer gelling agents, tissue including tissue wraps and tissue laminates, or any equivalent materials or combinations of materials. Suitable fluid storage layers comprising foams are described in European Applications 0 598 833, 0 598 823 and 0 598 834. Suitable fluid storage layers comprising tissue laminates with particles of superabsorbent or gelling agents comprised therebetween are described in International Patent Applications WO 94/01069 and WO 95/17868.

In one embodiment, odor controlling agents may be added to seek further reductions in odors. Such odor controlling agents include, but are not limited to activated charcoals, zeolites, silica, polyacrylic acids (superabsorbents), certain quaternary compounds, triethyl citrate, cyclodextrin, or any combinations thereof. Particularly preferred cyclodextrin compounds are described in U.S. Pat. No. 5,429,628 issued to Trihn et al. and U.S. Pat. No. 5,780,020 issued to Peterson et al. In addition, deodorants can be added to further mask these odors.

According to a particularly preferred embodiment of the present invention the absorbent element comprises only one layer, preferably one fluid storage layer. Any method known to those skilled in the art for coloring materials might be used too for coloring absorbent element, including pigmenting the material, dying the material and/or color printing the material.

d. Optional Fibrous Layer

An optional component for inclusion in the absorbent elements according to the present invention is a fibrous layer adjacent to, and typically underlying the storage layer. This underlying fibrous layer would typically provide the same function as the secondary fluid distribution layer.

e. Absorbent Article Design

The colored materials as indicated above can be used beneficially in the context of sanitary napkins, panty liners, and sweat pads (underarm or collar). A product design, which is a sub-form of a sanitary napkin or panty liner form, namely thong shaped sanitary napkins or panty liners, so called thong liners, are particularly susceptible to the present invention. The thong liner design is such that it provides the sanitary napkin or panty liner with a shape such that it can be worn in thong slips, G-string undergarments or string panties, hence the thong shape is fundamentally triangular or trapezoidal.

f. Optional Components of the Absorbent Articles

Optionally, the absorbent articles of the present invention can comprise all those components typical for the intended product use. For example absorbent articles can comprise components such as wings in order to improve their positioning and soiling protection performance especially towards the rear end of the article. Such designs are shown for example in EP 130 848 or EP 134 086, Thong liners with wings are shown in U.S. Design Pat. No. 394,503, UK Designs 2,076,491 and 2,087,071 as well as internationally filed industrial model DM 045544, filed under the Hague Agreement, registered on Oct. 21, 1998.

If present, the color of the wings are within the CIELab values. In fact an article design in which the wings are matching the value and preferably color of the remaining article is preferred. The reason is that wings when folded onto the external side of an undergarment are rather easily visible.

Irrespective whether the wings are specially designed for thong liners or for conventional absorbent articles they can be provided as separate pieces and be attached to the thong liner or conventional pantiliners or sanitary napkins, or they can be integral with the materials of the absorbent articles, e.g. by being integral extension of the topsheet, the backsheet or a combination thereof. If the wings are attached then they can be attached in a basic outward pointing position or already be predisposed towards their in-use position, i.e. towards the longitudinal centerline. If the wings are integral extensions of the topsheet or the backsheet or both then the color of the wings are provided within the required CIELab values.

In general, all typically used components in absorbent products can also be comprised in the absorbent articles according to the present invention as long as the absorbent articles meet the CIELab value criteria as set in the claims.

Most preferred absorbent articles will comprise a fastening adhesive for attachment. In the case of sanitary napkins, pantliners or thongliners a so-called panty fastening adhesive is preferred to be present on the backsheet for attachment to an undergarment. However, for sweat pads, e.g. underarm sweat pads, either attachment to an adjacent garment or attachment to the skin of the wearer directly can also be considered. Of course, such direct skin attachment, which is conventionally provided by water gel, hydrogel or oil gel based body adhesives, can also be used in sanitary napkins or body liners (in contrast to pantiliners).

### Test Method

Externally visible surfaces are tested in a dry state and at an ambient humidity of approximately 50%±2%. Reflectance color is measured using the Hunter Lab LabScan XE reflectance spectrophotometer obtained from Hunter Associates Laboratory of Reston, Va. The spectrophotometer is set to the CIELab color scale and with a D50 illumination. The Observer is set at 10° and the Mode is set at 45/0°. Area View is set to 0.125″ and Port Size is set to 0.20″ for films; Area View is set to 1.00″ and Port Size is set to 1.20″ for nonwovens and other materials. The spectrophotometer is calibrated prior to sample analysis utilizing the black and white reference tiles supplied from the vendor with the instrument. Calibration is done according to the manufacturer's instructions as set forth in LabScan XE User's Manual, Manual Version 1.1, August 2001, A60-1010-862. If cleaning is required of the reference tiles or samples, only tissues that do not contain embossing, lotion, or brighteners should be used (e.g., Puffs® tissue). Any sample point on the externally visible surface of the element containing the imparted color to be analyzed should be selected. Ideally, sample points are selected so as to be close in perceived color. A single ply of the element is placed over the spectrophotometer's sample port. A single ply, as used within the test method, means that the externally visible surface of the element is not folded. Thus, a single ply of an externally visible surface may include the sampling of a laminate, which itself is comprised of more than one lamina. The sample point comprising the color to be analyzed must be larger than the sample port to ensure accurate measurements. A white tile, as supplied by the manufacturer, is placed behind the externally visible surface. The L*, a*, and b* values are read and recorded. The externally visible surface is removed and repositioned so that a minimum of six readings are obtained for the externally visible surface. If possible (e.g., the size of the imparted color on the element in question does not limit the ability to have six discretely different, non-overlapping sample points), each of the readings is to be performed at a substantially different region on the externally visible surface so that no two sample points overlap. If the size of the imparted color region requires overlapping of sample points, only six samples should be taken with the sample points selected to minimize overlap between any two sample points. The readings are averaged to yield the reported L*, a*, and b* values for a specified color on an externally visible surface of an element.

In calculating the color space volume, V, maximum and minimum L*, a*, and b* values are determined for a particular set of elements to be color matched. The maximum and minimum L*, a*, and b* values are used to calculate V according to the formula presented above.

### Examples

The following is a listing of examples illustrating various embodiments of the present invention. It would be obvious to those skilled in the art that various other changes and modifications can be made without departing from the spirit and scope of the invention.

Examples 1-2, provided below, are sanitary napkins of the present invention. A sanitary napkin is to be tested having the same general construction as the ALWAYS® Regular sanitary napkin. Suitable sanitary construction is detailed in U.S. Pat. No. 5,489,283. The sanitary napkin of Example 1 and example 2 are substantially the same as those commercially available except for imparted colors on the backsheet, topsheet, and absorbent element. The backsheet is printed by flexographic printing. The topsheet is colored by color impregnation. The absorbent element is colored by dyeing.

The backsheet, the topsheet, and the absorbent element of the example are tested according to the test method described above. The three points tested (backsheet, the topsheet, and the absorbent element) fall within a color space volume of 97 according to the calculation described above.

Table 1 provides that three elements (backsheet, the topsheet, and the absorbent element) have a maximum ΔE* of 2.2. Calculation of ΔE* is performed on an element-to-element basis as described above. Given the three elements, three comparisons may be performed: backsheet, the topsheet, and the absorbent element. The ΔE* values for the three comparisons are 2.0, 2.1, and 2.2, respectively. The “-” in the Table indicates that element to element is not analyzed. In light of these values, the three elements are color matched in that all of the comparisons result in total color differences of less than about 2.2.

TABLE 1 ΔE*

| | Topsheet | Backsheet | Absorbent element |
|---|---|---|---|
| Topsheet | — | 2.0 | 2.2 |
| Backsheet | 2.0 | — | 2.1 |
| Absorbent | 2.2 | 2.1 | — |
| element | | | |

Table 2 provides that three elements (backsheet, topsheet, and absorbent element) have a maximum ΔH* of 0.9. Calculation of ΔH* is performed on an element-to-element basis as described above. Given the three elements, three comparisons may be performed: topsheet, backsheet, and absorbent element. The ΔH* values for the three comparisons are 0.6, 0.9, and 0.1, respectively. The “-” in the Table indicates that element to element is not analyzed. In light of these values, the three elements are color matched in that all of the comparisons result in hue difference of less than 0.9.

TABLE 2 ΔH*

| | Topsheet | Backsheet | Absorbent element |
|---|---|---|---|
| Topsheet | — | 0.6 | 0.9 |
| Backsheet | 0.6 | — | 0.1 |
| Absorbent | 0.9 | 0.1 | — |
| element | | | |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Detailed Description are in relevant part incorporated herein by reference; the citation of any document is not to be construed as an admission that it is prior art with respect to the present invention.

What is claimed is:

1. A disposable absorbent article, comprising:
a. a liquid pervious topsheet;
b. a liquid impervious backsheet comprising adhesive for position against an internal side of an undergarment;
c. a pair of wings for position against an external side of an undergarment;
d. a first imparted color disposed on the topsheet; and
e. a second imparted color disposed on the pair of wings;
f. wherein the first imparted color is matched to the second imparted color so that the first imparted color and the second imparted color are within a CIELab color space volume of less than about 190.

2. The disposable absorbent article of claim 1, wherein the topsheet comprises an apertured formed film.

3. The disposable absorbent article of claim 1, wherein the topsheet comprises an apertured nonwoven.

4. The disposable absorbent article of claim 1, wherein the first imparted color and the second imparted color have a CIELab C* value of greater than 6.

5. The disposable absorbent article of claim 1, wherein at least one of the first imparted color and the second imparted color is selected from the group consisting of orange, purple, lavender, red, green, blue, yellow, and violet.

6. The disposable absorbent article of claim 1, wherein the first imparted color and the second imparted color are imparted by printing.

7. The disposable absorbent article of claim 1, wherein the article is a sanitary napkin.

8. A disposable absorbent article, comprising:
a. a liquid pervious topsheet;
b. a liquid impervious backsheet comprising adhesive for position against an internal side of an undergarment;
c. a pair of wings for position against an external side of an undergarment;
d. a first imparted color disposed on the topsheet; and
e. a second imparted color disposed on the pair of wings;
f. wherein the first imparted color is matched to the second imparted color so that the first imparted color and the second imparted color are within a CIELab color space hue difference of less than about 4.

9. A disposable absorbent article, comprising:
a. a liquid pervious topsheet;
b. a liquid impervious backsheet comprising adhesive for position against an internal side of an undergarment;
c. a pair of wings for position against an external side of an undergarment;
d. a first imparted color disposed on the topsheet; and
e. a second imparted color disposed on the pair of wings;
f. wherein the first imparted color is matched to the second imparted color so that the first imparted color and the second imparted color are within a CIELab total color difference of less than about 3.

10. A disposable absorbent article, comprising:
a. a liquid pervious topsheet;
b. a liquid impervious backsheet;
c. adhesive disposed on the backsheet;
d. a release liner covering the adhesive;
e. a first imparted color disposed on the topsheet; and
f. a second imparted color disposed on the release liner;
g. wherein the first imparted color is matched to the second imparted color so that the first imparted color and the second imparted color are within a CIELab color space volume of less than about 190.

11. The disposable absorbent article of claim 10, wherein the topsheet comprises an apertured formed film.

12. The disposable absorbent article of claim 10, wherein the topsheet comprises an apertured nonwoven.

13. The disposable absorbent article of claim 10, wherein the first imparted color and the second imparted color have a CIELab C* value of greater than 6.

14. The disposable absorbent article of claim 10, wherein at least one of the first imparted color and the second imparted color is selected from the group consisting of orange, purple, lavender, red, green, blue, yellow, and violet.

15. The disposable absorbent article of claim 10, wherein the first imparted color and the second imparted color are imparted by printing.

16. The disposable absorbent article of claim 10, wherein the article is a sanitary napkin.

17. A disposable absorbent article, comprising:
a. a liquid pervious topsheet;
b. a liquid impervious backsheet;
c. adhesive disposed on the backsheet;
d. a release liner covering the adhesive;
e. a first imparted color disposed on the topsheet; and
f. a second imparted color disposed on the release liner;
g. wherein the first imparted color is matched to the second imparted color so that the first imparted color and the second imparted color are within a CIELab color space hue difference of less than about 4.

18. A disposable absorbent article, comprising:
a. a liquid pervious topsheet;
b. a liquid impervious backsheet;
c. adhesive disposed on the backsheet;
d. a release liner covering the adhesive;
e. a first imparted color disposed on the topsheet; and
f. a second imparted color disposed on the release liner;
g. wherein the first imparted color is matched to the second imparted color so that the first imparted color and the second imparted color are within a CIELab total color difference of less than about 3.

19. A disposable absorbent article, comprising:
a. a liquid pervious topsheet;
b. a liquid impervious backsheet;
c. adhesive disposed on the backsheet;
d. a release liner covering the adhesive;
e. a first imparted color disposed on the backsheet; and
f. a second imparted color disposed on the release liner;
g. wherein the first imparted color is matched to the second imparted color so that the first imparted color and the second imparted color are within a CIELab color space volume of less than about 190.

20. The disposable absorbent article of claim 19, wherein the topsheet comprises an apertured formed film.

21. The disposable absorbent article of claim 19, wherein the topsheet comprises an apertured nonwoven.

22. The disposable absorbent article of claim 19, wherein the backsheet comprises a nonwoven and polymeric film composite.

23. The disposable absorbent article of claim 19, wherein the first imparted color and the second imparted color have a CIELab C* value of greater than 6.

24. The disposable absorbent article of claim 19, wherein at least one of the first imparted color and the second imparted color is selected from the group consisting of orange, purple, lavender, red, green, blue, yellow, and violet.

25. The disposable absorbent article of claim 19, wherein the first imparted color and the second imparted color are imparted by printing.

26. The disposable absorbent article of claim 19, wherein the article is an incontinence pad.

27. A disposable absorbent article, comprising:
a. a liquid pervious topsheet;
b. a liquid impervious backsheet;
c. adhesive disposed on the backsheet;
d. a release liner covering the adhesive;
e. a first imparted color disposed on the backsheet; and
f. a second imparted color disposed on the release liner;
g. wherein the first imparted color is matched to the second imparted color so that the first imparted color and the second imparted color are within a CIELab color space hue difference of less than about 4.

28. A disposable absorbent article, comprising:
a. a liquid pervious topsheet;
b. a liquid impervious backsheet;
c. adhesive disposed on the backsheet;
d. a release liner covering the adhesive;
e. a first imparted color disposed on the backsheet; and
f. a second imparted color disposed on the release liner;
g. wherein the first imparted color is matched to the second imparted color so that the first imparted color and the second imparted color are within a CIELab total color difference of less than about 3.
